(19) United States
(12) Patent Application Publication
(10) Pub. No.: US 2018/0333357 A1
(43) Pub. Date: Nov. 22, 2018

(54) NANOEMULSIONS WITH ANTI-INFLAMMATORY ACTIVITY

(71) Applicant: Ocugen, Inc., Malvern, PA (US)

(72) Inventors: Rasappa Arumugham, Lansdale, PA (US); Arun K. Upadhyay, West Chester, PA (US)

(73) Assignee: Ocugen, Inc., Malvern, PA (US)

(21) Appl. No.: 15/963,955

(22) Filed: Apr. 26, 2018

Related U.S. Application Data
(63) Continuation-in-part of application No. 15/946,709, filed on Apr. 5, 2018.
Application No. 15/946,709 is a continuation-in-part of application No. PCT/US18/26342, filed on Apr. 5, 2018.
(60) Provisional application No. 62/509,015, filed on May 19, 2017.
(60) Provisional application No. 62/591,548, filed on Nov. 28, 2017.

(51) Int. Cl. A61K 9/107; A61K 47/44; A61K 47/32; A61K 47/26; A61K 47/10; A61K 9/00; A61P 27/02

(52) U.S. Cl. CPC A61K 9/1075; A61K 47/44; A61K 47/32; A61P 27/02; A61K 47/10; A61K 9/0048; A61K 47/26

(57) ABSTRACT

A sterile-filtered nanoemulsion having oil globules with a median size (Dv50) of about 100 nm or less and containing an oil, emulsion stabilizing polymer, a water-soluble polymer a surfactant, a tonicity modifier or stabilizer, or a combination thereof is provided. The nanoemulsion, even in the absence of an active ingredient, has ameliorative effects including anti-inflammatory activity. The present invention also provides a method of using such a nanoemulsion as an artificial tear composition for treating an ocular surface disorder, including those resulting from contact lens use, by administering an effective amount of the oil-in-water nanoemulsion of the present invention topically to an eye of a subject.

### CROSS-REFERENCE TO RELATED APPLICATIONS

This application is a continuation-in-part application of U.S. patent application Ser. No. 15/946,709, filed Apr. 5, 2018, and a continuation-in-part application of PCT Patent Application No. PCT/US18/26342, filed Apr. 5, 2018, both of which claim priority benefit of U.S. Provisional Application Nos. 62/509,015, filed May 19, 2017, and 62/591,548, filed Nov. 28, 2017, all of which are incorporated herein by reference in their entirety.

### FIELD OF THE INVENTION

The present invention relates to a sterile-filtered nanoemulsion having oil globules with a median size (Dv50) of about 100 nm or less, such as in the range of from about 40 to about 80 nm, and having an anti-inflammatory activity even in the absence of an active ingredient in the formulation. The present invention also relates to method of making the nanoemulsion and to the use of the nanoemulsion for topical application for a number of purposes in particular as an artificial tear.

### BACKGROUND OF THE INVENTION

In the last decade, oil-in-water-type lipid emulsions (e.g., nanoemulsions), primarily intended for parenteral applications, have been investigated and are now being exploited as a vehicle to deliver lipophilic drug substances for cosmetic, dermatological and ophthalmological purposes. In the ophthalmological field, dry eye disease is the most actively investigated area.

Clinicians around the world recognize the necessity to treat “dry eye patients” in a comprehensive way, taking into account their symptoms, meibomian gland physiology, tear film lipid quality and quantity, meibomian gland orifice patency, and also tear production, loss and runoff.

Historically, dry eye disease (DED) was considered to be largely due to tear insufficiency and was treated by prescribing tear replacement products or by conserving the tears via punctal plugs. More recent treatments have included the use of methods to stimulate tears.

Tear replacement with ocular lubricants is traditionally considered a mainstay of DED therapy and there are numerous topical formulations available. Over-the-counter (OTC) products are often termed “artificial tears” which, as their name suggests, attempt to replace and/or supplement the natural tear film (TF). Typical treatments for dry eye syndrome include (i) instillation of artificial tears for tear supplementation and stimulation and (ii) the use of anti-inflammatory drugs to reduce ocular surface inflammation. Generally, current dry eye treatment involves topical application of artificial tear products/lubricants, tear retention management, stimulation of tear secretion, topical application of antibiotics (e.g., erythromycin or bacitracin ointments), oral administration of tetracyclines (e.g., tetracycline, doxycycline, or minocycline), application of anti-inflammatory compounds (cyclosporin) and corticosteroids. These treatments are often time consuming, frustrating, and frequently ineffective or variably effective.

The nanosize (submicron-size, i.e., <1 μm) of the oil droplets (the interfacial area available for drug exchange) creates a huge contact surface with the ocular surface cells enabling enhanced absorption. Cationorm® (CN), a cationic nanoemulsion has been reported to enhance TF stability in vivo. See Georgieve et al., 2017, Int. J. Mol. Sci., 18:1558. Such cationic emulsions were observed to have an inherent benefit on the ocular surface in terms of reduced symptoms of ocular surface even in the absence of an active ingredient. The success of the CN was attributed to the interaction between the positively charged formulation and negatively charged corneal cells. Lallemand et al., 2012, J Drug Deliv., 2012:1-16. The first marketed ophthalmic emulsion drug product was Restasis (Allergan), a preservative-free anionic emulsion of cyclosporine A (CsA) at 0.05% indicated to increase tear production in patients whose tear production is presumed to be suppressed due to ocular inflammation. Other emulsion-based eye drops available on the market are artificial tears Refresh Endura (Allergan), Soothe (Bausch & Lomb), etc. Another product for treating dry eye syndrome is lifitegrast (commercially available as Xiidra® ophthalmic solution 5%, Shire US Inc., Lexington, Mass.). Lifitegrast (chemical name: N-{[2-(1-Benzofuran-6-ylcarbonyl)-5,7-dichloro-1,2,3,4-tetrahydro6-isoquinolinyl]carbonyl}-3-(methylsulfonyl)-L-phenylalanine) ophthalmic solution 5.0% has been reported to improve symptoms of ocular discomfort and eye dryness compared with placebo when administered twice daily (Sheppard et al., Ophthalmology, 2014, 121(2), pp. 475483).

Ophthalmic products, especially multidose products typically require a preservative to prevent microbial growth. The concentration of a preservative in an ophthalmic product should be a compromise between preservative efficacy and toxicity. An alternative to the preservative use could be to subject the product to sterilization process so the product is sterile. It is known in the art from Lallemand et al., 2012, J Drug Deliv., 2012:1-16) that (i) a sterilizing filtration is not possible for emulsions as it uses a filter with 0.22 μm size pores that can clog during filtration and (ii) aseptic processes are too expensive. The remaining option was heat sterilization which has its own limitations. Hence the need for a careful choice of cationic agents for nanoemulsion has emerged. These cationic agents are benzalkonium chloride, cetalkonium chloride, cetylpyridinium chloride, cetrimide and benzethonium chloride. However, these are all known preservative or antibacterial compounds.

The need, therefore, remains for nanoemulsions which do not have the disadvantages of those of the prior art. In particular, there is a need for sterile filtered nanoemulsion formulations without active pharmaceutical ingredients in them for effective relief from various disorders including dry eye syndrome and methods for making and using such nanoemulsions.

### SUMMARY OF THE INVENTION

It has now been found that, surprisingly and unexpectedly, certain nanoemulsions even without any active pharmaceutical ingredient (“API”) in them have anti-inflammatory properties on topical application to mammals thereby making it possible to obtain ophthalmic compositions without any API's but exhibiting all the advantages of known ophthalmic API formulations, such as those described above, without their disadvantages. In some embodiments, unlike Cationorm®, the nanoemulsion formulations of the invention do not include any cations. In some instances, the nanoemulsion formulations of the invention are neutral, meaning there is no anion or cation in the nanoemulsion. Without being bound by any theory, it is believed that the nanoemulsion formulation of the invention provides observed clinical activity primarily via a lipid-lipid interaction.

Accordingly, in an aspect, the present invention relates to an oil-in-water nanoemulsion having oil globules with a median size (Dv50) of about 100 nm or less. Preferably, the oil globules have a median size (Dv50) ranging from about 20 to about 80 nm. Still more preferably, the oil globules have a median size (Dv50) ranging from about 40 to about 60 nm. The oil-in-water nanoemulsion contains an oil, and at least one or more of emulsion stabilizing polymers, a water-soluble polymer, a surfactant, a tonicity modifier or stabilizer. The nanoemulsion is a sterile-filtered nanoemulsion and is optionally preservative-free. The nanoemulsion, even in the absence of an active ingredient, has ameliorative effects comprising anti-inflammatory activity on the ocular surface as determined by an improved corneal fluorescein staining (CFS) or CFS score compared to a therapeutic pharmaceutical composition containing lifitegrast. In one particular embodiment, an oil-in-water nanoemulsion comprises oil globules with a median size (Dv50) of about 100 nm or less. In certain instances, said nanoemulsion is a sterile-filtered nanoemulsion. Yet in other instances, the oil-in-water nanoemulsion consists essentially of an oil, an emulsion stabilizing polymer, a water soluble polymer, a surfactant, a tonicity modifier or stabilizer, a buffer, water, or a combination thereof.

In one embodiment, the oil in the emulsion is selected from castor oil, corn oil, olive oil or oleic acid, sesame oil, soybean oil, cottonseed oil, peanut oil, safflower oil, sunflower oil, palm oil, palm kernel oil, and canola oil or a combination thereof. Preferably the oil is castor oil. Preferred surfactant is polysorbate 80, and Pemulen® used as an emulsifier. Tonicity modifier or stabilizer is selected from a polyol, a non-reducing disaccharide, and a combination thereof.

Preferably the oil-in-water nanoemulsion contains glycerin, a polymeric emulsifier (e.g., Acrylates/C₁₀₋₃₀ Alkyl Acrylate Cross-Polymer or Pemulen®), castor oil, a surfactant (e.g., polysorbate 80) and a buffering salt (e.g., sodium citrate dehydrate), and has a pH in the range of from about pH 5 to about pH 8. Preferably glycerin is present at a concentration of about 2.2% w/w, the polymeric emulsifier is present at a concentration of about 0.05% w/w, castor oil is present at a concentration of about 1.25% w/w, the surfactant is present at a concentration of about 1% w/w, and the buffering salt is present at a concentration of about 0.15% w/w. Sufficient quantity of pH adjusting solutions are preferably added to prepare final nanoemulsion formulation. This final formulation is sterilized preferably by sterile filtration.

In another embodiment, the oil-in-water nanoemulsion is part of a vehicle for topically delivering a cosmetically, dermatologically or ophthalmologically active pharmaceutical agent.

In another aspect, the present invention relates to a method of preparing the oil-in-water nanoemulsion described above. The method entails, among other steps, mixing the aqueous phase and the oil phase with vigorous stirring at a temperature ranging from about 55° C. to about 65° C., high shear mixing of the aqueous phase and the oil phase using homogenization method at a temperature ranging from about 55 to about 65° C. to realize a homogenized mixture. The homogenizing of the mixture is conducted at 3000 to 15000 rpm under high shear. The homogenized mixture is further subjected to high pressure preferably through a microfluidization method. Subjecting the homogenized mixture to a further high pressure is preferably carried out at a temperature ranging from about 55 to about 65° C. and at a pressure ranging from about 20000 to about 30000 psi.

In another aspect, the present invention relates to a method of treating ocular surface disorders. “Treating” or “treatment” of an ocular disorder includes: (1) preventing the disease, i.e., causing the clinical symptoms of the disease not to develop in a subject that may be exposed to or predisposed to the ocular disorder but does not yet experience or display symptoms of the ocular disorder; (2) inhibiting, arresting, ameliorating, or reducing the development of the ocular disorder or its clinical symptoms; or (3) relieving the ocular disorder or the symptoms of ocular disorder, i.e., causing regression of the ocular disorder or its clinical symptoms. The method involves administering an effective amount of the oil-in-water nanoemulsion of the present invention (i.e., “NanoE” or “ONE™”) topically to an eye of a subject (e.g., a human) in need of such a treatment. In one particular embodiment, the ocular surface disorder is a dry eye syndrome.

In another aspect, the present invention relates to an oil-in-water nanoemulsion that is sterile-filterable. The nanoemulsion is preferably preservative-free. The nanoemulsion formulation of the invention can be prepared by a process involving mixing the aqueous phase and the oil phase with vigorous stirring typically at a temperature ranging from about 55 to about 65° C., homogenizing the mixture typically at a temperature ranging from about 55 to about 65° C. at 3000-15000 rpm and subjecting the homogenized mixture to high pressure shearing typically at a pressure ranging from about 20000 to about 30000 psi to provide the oil-in-water nanoemulsion of the invention. The oil-in-water nanoemulsion so realized can optionally be filtered using a 0.22 μm filter. Typically, the oil globules in nanoemulsion formulations of the invention have a median size (Dv50) ranging from about 20 to about 80 nm, and often ranging from about 40 to about 60 nm. The sterile filtered nanoemulsion even in the absence of an active ingredient has therapeutic or ameliorative effects such as anti-inflammatory activity particularly on the ocular surface as determined by an improved corneal fluorescein staining (CFS). Surprisingly and unexpectedly, it was found that in some embodiments, the nanoemulsion formulations of the invention has anti-inflammatory activity similar to or better than a therapeutic pharmaceutical composition containing lifitegrast. As used herein, unless the context requires otherwise, the terms “active pharmaceutical ingredient” and “active ingredient” are used interchangeably herein and refers to a drug or a physiologically active compound approved or as recognized by the Food and Drug Administration (“FDA”) or a compound recognized by one skilled in the art as being an active ingredient, see for example, en.wikipedia.org/wiki/Active_ingredient. In one particular embodiment, the nanoemulsion formulations of the invention do not include any art recognized active ingredient.

In another aspect of the invention, an artificial tear composition containing the oil-in-water nanoemulsion of the present invention and method of use of such artificial tears are provided. In an embodiment, the oil-in-water nanoemulsion by itself is used as an artificial tear in which instance the artificial tears of the invention consist of the oil-in-water nanoemulsion of the present invention. The artificial tear composition of the invention can also consist essentially of the oil-in-water nanoemulsion of the present invention, i.e., it necessarily includes the oil-in-water nanoemulsion of the present invention, and is open to other components or ingredients (exclusive of any active pharmaceutical ingredients) that do not materially affect the basic and novel properties of the artificial tear composition of the invention. Preferably, the artificial tear composition containing the oil-in-water nanoemulsion of the present invention or consisting of or consisting essentially of the oil-in-water nanoemulsion is used in a process for treating the symptoms of ocular surface disorders such as dry eye or from contact lens use. The method involves administering topically to an eye of a human subject in need of such a treatment an effective amount of the artificial tear containing, consisting of or consisting essentially of the oil-in-water nanoemulsion of the present invention.

### BRIEF DESCRIPTION OF THE DRAWINGS

FIG. 1 is a graph showing Particle Size Distribution (PSD) of nanoemulsion measured using Mastersizer 3000 instrument (Malvern, UK).

FIG. 2A shows images depicting representative corneal fluorescein staining (CFS) scores from the mouse dry-eye disease model in the three different groups from left to right as follows: (A) untreated; (B) NanoE nanoemulsion formulation (NanoE) only; and (C) Xiidra® (lifitegrast ophthalmic solution) 5% lifitegrast.

FIG. 2B is a histogram showing data related to the effect of placebo nanoemulsion against corneal epithelial damage in different groups as described in FIG. 2A. Data are presented as median±interquartile range.

### DETAILED DESCRIPTION OF THE INVENTION

This invention is based, at least in part, on the discovery by the present inventors that the nanoemulsions of the invention (“NanoE” or “ONE™”), even in the absence of an active pharmaceutical ingredient, have a surprising and unexpected benefit on the ocular surface in terms of, including but not limited to, its anti-inflammatory activity. In an aspect of the invention, a nanoemulsion, preferably an oil-in-water nanoemulsion with an oil phase dispersed in an aqueous phase, having an anti-inflammatory activity is provided. The nanoemulsions of the present invention are free of active pharmaceutical ingredient(s). The term “active pharmaceutical ingredient” is well known to one skilled in the art and is defined as any substance or mixture of substances intended to be used in the manufacture of a drug product and that, when used in the production of a drug, becomes an active ingredient in the drug product. The nanoemulsion of the present invention can be used as a topical ophthalmic formulation. In some embodiments, the nanoemulsion of the present invention is used as an artificial tear composition.

The formulation necessarily contains an oil or a fatty acid ester. A fatty acid ester has the meaning commonly understood in the art, being an ester formed between an alcohol and a fatty acid. Exemplary fatty acid esters that are useful in formulations of the invention include, but are not limited to, triglyceride esters commonly known as vegetable oils, mono and diglyceride esters of fatty acids, fatty acid methyl esters, as well as other fatty acid esters that are known to one skilled in the art. It should be appreciated the fatty acid ester can be a mixture of several chemical compounds or an essentially pure compound. Typically, the fatty acid ester is a vegetable oil. Particular examples of vegetable oils that can be used include, but are not limited to, castor oil, corn oil, olive oil, oleic acid, sesame oil, soybean oil, cottonseed oil, peanut oil, safflower oil, sunflower oil, palm oil, palm kernel oil, and canola oil. Preferably the formulation contains castor oil, corn oil, olive oil, oleic acid or a combination of these components. It is particularly preferred that the fatty acid ester is castor oil. The oil phase can also contain fatty substances other than the oils indicated above, such as fatty alcohols, for example, stearyl, cetyl and behenyl alcohols, fatty acids, for example stearic, palmitic and behenic acids, oils of fluorinated type, waxes, gums and their mixtures.

Besides an oil or a fatty acid ester, the formulation can contain a pharmaceutically acceptable excipient including an emulsion stabilizing polymer, a surfactant, a water-soluble polymer, a tonicity modifier or a stabilizer selected from the group consisting of a polyol, a non-reducing disaccharide, and a combination thereof, buffering salts, and pH adjusting acid/base solutions. While not intending to limit the scope of the invention, emulsion stabilizing polymers generally contain hydrophilic groups such as cellulose, sugars, ethylene oxide, hydroxide, carboxylic acids or other polyelectrolytes. Without being bound by any theory, it is believed that these polymers help to stabilize emulsions. Some examples of emulsion stabilizing polymers useful in this invention include, but are not limited to, carbomers, Pemulen®, sodium carboxymethylcellulose, hydroxypropylmethylcellulose, povidone, polyethylene glycol and a mixture of two or more thereof.

In some embodiments, Pemulen® (B.F. Goodrich, Cleveland, Ohio) is used as the polymeric emulsifier and/or nanoemulsion stabilizer. Pemulen® are Acrylates/C₁₀₋₃₀ Alkyl Acrylate Cross-Polymers.

A number of water-soluble polymers are known in the art. Preferably for the nanoemulsion of the present invention, the water-soluble polymer can be any of polyacrylamides, polyacrylic acids or copolymers of polyacrylic acid, polyethylene oxides, guars, hydroxyethyl celluloses, polyvinyl alcohols or mixtures thereof. Preferably, the water-soluble polymer is nonionic, that is to say neutral. Preferably, the water-soluble nonionic polymer is present in a nanoemulsion thickening effective amount.

The formulation of this invention further contains a surfactant. Without being bound by any theory, a surfactant is used to help facilitate the formation of the emulsion and improve its stability. Any type of surfactant can be used including, anionic, amphoteric, zwitterionic, nonionic, as well as a mixture of two or more thereof. Preferably, the formulation of the invention contains an anionic or a nonionic surfactant. Exemplary nonionic surfactants include, but are not limited to, polysorbates, poloxamers, alcohol ethoxylates, ethylene glycol-propylene glycol block copolymers, fatty acid amides, alkylphenol ethoxylates, phospholipids, and two or mixture thereof. In one particular embodiment, the surfactant is Polysorbate 80. The formulation of the present invention is preferably free of any cationic surfactant or contains surfactants other than cationic surfactants. Preferably the formulation contains surfactants that are nonionic. Preferably, the nanoemulsion of the present invention containing nano-size globules contains castor oil, a surfactant-polysorbate 80, and an emulsifier-Pemulen TR2 and a tonicity agent-glycerine. Preferred formulation has the nano-size globules that are neutral at formulation pH (pH range of from about pH 5 to about pH 8), preferably at physiological pH (e.g., pH 7.4) without any significant positive or negative surface charges.

The amount of surfactant in the nanoemulsion of the invention can range, for example, from about 0.1 to about 40 mg (i.e., 0.01% to 4% by weight) and preferably from about 0.5% to about 1% by weight with respect to the total weight of the nanoemulsion. These ranges include all specific values and subranges therebetween, such as 0.2, 0.3, 0.4, 0.5, 0.6, 0.7, 0.8, 0.9 and 1.0%.

Preferably, a tonicity agent (tonicity-adjusting agent) is used to adjust the composition of the formulation to the desired isotonic range. The tonicity-adjusting agent can be a polyol or a disaccharide including non-reducing disaccharides. Such tonicity agents are known to one skilled in the art, and include, but are not limited to, glycerin, mannitol, sorbitol, trehalose, xylitol, sodium chloride, and other electrolytes. In one particular embodiment, the tonicity agent is glycerin.

If desired, gum and/or resin can be included in the formulations of the invention, including for example, sodium polyacrylate, cellulose ether, calcium alginate, carboxyvinyl polymer, ethylene-acrylic acid copolymer, vinyl pyrrolidone polymer, vinyl alcohol-vinyl pyrrolidone copolymer, nitrogen-substituted acrylamide polymer, polyacrylamide, cationic polymer such as cationic guar gum, dimethylacrylic ammonium polymer, acrylic acidmethacrylic acid copolymer, polyoxyethylene-polypropylene copolymer, polyvinyl alcohol, pullulan, agar, gelatine, chitosan, polysaccharide from tamarindo seed, xanthan gum, carageenan, high-methoxyl pectin, low-methoxyl pectin, guar gum, acacia gum, microcrystalline cellulose, arabinogalactan, karaya gum, tragacanth gum, alginate, albumin, casein, curdlan, gellan gum, dextran, cellulose, polyethyleneimine, high polymerized polyethylene glycol, cationic silicone polymer, synthetic latex, acrylic silicone, trimethylsiloxysilicate and fluorinated silicone resin.

In certain embodiments, the formulations of the present invention are directed to artificial tear compositions containing combinations components but without any exogenously supplied active pharmaceutical ingredients in them (without regard to the use of transitional phrases such as “comprising” in claiming the invention herein) whereby an oil or a fatty acid ester, one or more nonionic surfactants and/or at least one excipient selected from polysorbate 80, an emulsion stabilizing polymer (e.g., Acrylates/C₁₀₋₃₀ Alkyl Acrylate Cross-Polymer or) Pemulen®, a polyol or a combination thereof confer the desired properties of relief from ocular disorders including those from contact lens use. The present invention is based, in part, on the surprising discovery that nanoemulsion of the present invention without any added active pharmaceutical ingredients can be used as artificial tears as a first-line therapy for ocular surface disorders such as dry eye or from contact lens use. Without wishing to be bound by any theory, it is believed that the artificial tears containing nanoemulsion of the present invention improve or ameliorate the symptoms of ocular surface disorders by lubricating the ocular surface to create an evaporative tear shield that can stabilize the aqueous and lipid layers of the tear film and/or by increasing tear volume. Artificial tear compositions of the present invention also modestly confer anti-inflammatory activity on the ocular surface. The net effect of the relief provided by the artificial tear compositions of the present invention is at least as effective as, if not greater than, that provided by prescription medications such as Xiidra® ophthalmic solution 5% made by Shire US Inc., Lexington, Mass. Artificial tears can be formulated containing the oil-in-water nanoemulsion of the present invention. Accordingly, an artificial tear composition containing, consisting essentially of or consisting of the oil-in-water nanoemulsion of the present invention is used in a method for improving or ameliorating the symptoms of ocular surface disorders such as dry eye or from contact lens use.

In another aspect, the invention provides methods of preparing the formulations of the present invention. Preferably, the formulation is a nanoemulsion. The manufacturing process involves phase mixing of the aqueous phase and the oil phase. The mixture is preferably stirred at 100-200 rpm or higher for several minutes to hours. After phase mixing is complete, the samples are subjected to high shear mixing under homogenization. The shearing preferably ranges from 3000 to 15000 rpm and at 55 to 65° C. Preferably, the high shear mixing is carried out at 3000-15000 rpm for 5-15 min depending on the manufacturing scale. High shear mixing is carried out to realize or obtain globules in the size range of <10 micron (or μ), preferably <7 micron, more often <5 micron. In order for achieving submicron sizing, preferably below 100 nm, of the oil droplets, the emulsion is put through a high-pressure microfluidization by passing the mixture through the high-pressure microfluidizer, preferably at a pressure higher than 20000 psi, at an ambient temperature of 45° C.-65° C. Preferably the high-pressure homogenization is carried out at a pressure ranging from 20000 to 30000 psi. Such a process makes it possible to obtain nanoemulsions that can be filtered through a 0.22 μm filter without clogging the sterile filter during filtration. Preferably the process results in globule mean size (Dv 50) of ≤150 nm, or ≤100 nm, or between 40-80 nm or 40-60 nm. According to Malvern Mastersizer 3000 instrument used to measure size distribution in the instant case, Dv 50 is the size in microns at which 50% of the sample is smaller and 50% is larger than this diameter. This value is also known as the Mass Median Diameter (MMD) or the median of the volume distribution. The v in the expression Dv 50 refers to the volume distribution. The viscosity can be in the range of 1-10 cP or cps when measured at ambient temperature (e.g., 25° C. or 32° C. or lower). The viscosity can be measured by any of a number of viscometers known in the art such as, for example, Brookfield viscometer (e.g., Model-DV2TLVCJ) and Spindle (e.g., CP-52) at 20 rpm. The final nanoemulsions can be filtered through a 0.22 μm filter without clogging during filtration. In some embodiments, formulations or nanoemulsions of the present invention are sterilized by filtering through a 0.22 μm filter (i.e., sterile-filtered) unless otherwise stated as sterilized by other means such as, for example, by autoclaving at about 121° C., or by gamma or e-beam irradiation, or by using antimicrobials or preservatives, etc.

Preferably, a method of preparing the oil-in-water nanoemulsion can include mixing the aqueous phase and the oil phase with vigorous stirring at a temperature ranging from 55 to 65° C., homogenizing the mixture at a temperature ranging from 55 to 65° C. at 3000-15000 rpm under high shear and a further high pressure shearing using a microfluidizer at a much higher pressure which can range preferably from 20000 to 30000 psi. The step of homogenizing the mixture is preferably carried out at 3000-15000 rpm under high shear.

One particular embodiment of a process flow for preparing a nanoemulsion of the invention is described below:
1. Mix Oil phase: Mix appropriate amounts of castor oil and polysorbate 80 until uniformity is obtained.
2. Mix Aqueous phase: Mix required amounts of Pemulen, water and glycerin until uniformity is obtained.
3. Perform primary mixing of oil and aqueous phase mixtures from steps 1 and 2.
4. Perform high shear mixing using homogenization of mixture from step 3.
5. Perform high-pressure microfluidization using microfluidizer.
6. Confirm the nanoemulsion properties via in-process testing.
7. Add required amount of other excipients, like glycerin, buffer salts and pH adjusting solution to prepare final nanoemulsion formulation. Perform sterile filtration.

The above steps of the process flow need not be carried out in the same order.

An example of a nanoemulsion with its various components (w/w) is as follows: surfactant such as Polysorbate 80 at about 0.02%-2% by weight or poloxamer/tyloxapol at about 0.1% and 0.25% by weight; carbomer copolymer (type A or type B) about 0.05% by weight; tonicity agent (glycerine or includes glycerine about 2.2% by weight; citrate/tris buffer of pH 6.0-8.0, sodium EDTA in the amount of about 0.02% or less by weight; an oil (e.g., castor oil) in the amount of about 1.25% by weight. Alternatively, the oil for the oil phase is a medium chain triglyceride in the range from 0.5-4%, typically at about 2%. To prepare this formulation, all water-soluble components can be added and heated (about 60-70° C.) to make water the phase with buffer. A lipophilic solution is prepared using a lipophilic solvent (e.g., castor oil) and heating to about 60-70° C. The nanoemulsion is formed by rapid addition of lipophilic solution into water phase followed by high shear mixing. The final solution is sterilized via 0.22 micron filter. Alternatively, sterilization can also be done by autoclaving at about 121° C. for 20 min. Alternatively, sterilization can also be done by gamma or e beam irradiation. The sterilized nanoemulsion is filled into single dose disposable tubes by BFS technology or the like or into multi-dose container/closure systems (further described elsewhere herein). The formulation can be used topically, for caring for cosmetic, dermatological or ophthalmological conditions. Preferably, this and other compositions described throughout the disclosure herein can be used for treating an eye disorder (e.g., dry eye syndrome) or alleviating the symptoms of the eye disorder or as an artificial tear composition.

Yet another example of the present invention formulation with its various components (w/w) is as follows. This particular formulation includes carbomer homopolymer type B in an amount ranging from about 0.2% to about 0.6%, by weight typically in an amount of about 0.4% or about 0.25%, and/or carbomer homopolymer type C in an amount ranging from about 0.4 to about 5% typically in an amount of about 4% or about 2.5%, and/or polycarbophil in an amount ranging from about 0.2% to about 0.5% typically in an amount of about 0.4% or about 0.2%; glycerin in an amount ranging from about 0.5% to about 1% typically in an amount of about 0.9%; optionally, benzalkonium chloride in an amount ranging from about 0.003% to about 0.01% typically in an amount of about 0.007%; edetate sodium in an amount ranging from about 0.03% to about 0.07% typically in an amount of about 0.05%; sodium chloride in an amount of up to about 0.09%, typically in an amount of about 0.06% or q.s. to isotonicity, or mannitol q.s. to isotonicity, or without isotonicity adjustors sodium chloride and mannitol; propylene glycol in an amount ranging from about 0.3% to about 0.6% typically in an amount of about 0.5%; water q.s., to 100 g and sodium hydroxide or hydrochloric acid q.s., to adjust pH to 7.8. Although preservatives such as benzalkonium chloride can be used in the formulations of the present invention as described in the non-limiting examples, the formulations of the present invention are preferably preservative-free unless stated otherwise. In the context of the present invention, a “preservative” is a substance or a chemical that is added to products such as pharmaceutical and dermatological products and cosmetics and many other products in an attempt to prevent decomposition by microbial growth or by undesirable chemical changes. The term “preservative-free” used in connection with the nanoemulsions of the present invention refers to the formulations free of preservatives including benzalkonium chloride, cetalkonium chloride, cetylpyridinium chloride, cetrimide and benzethonium chloride and such other known preservatives. The formulation can be used topically, for caring for cosmetic, dermatological or ophthalmological conditions. Preferably, it can be used for treating an eye disorder (e.g., dry eye syndrome) or alleviating the symptoms of the eye disorder or as an artificial tear composition.

Another example of a formulation with its various components (w/w) is as follows: a surfactant such as Polysorbate 80 at about 0.02%-2% by weight or carbomer copolymer (type A or type B) about 0.05% by weight; tonicity agent (glycerine or includes glycerine about 2.2% by weight; sodium citrate and tris buffer of pH 6.0-8.0, sodium EDTA in the amount of about 0.02% or less by weight; an oil (e.g., castor oil) in the amount of about 1.25% by weight.

Another example of a formulation with its various components (w/w) is as follows: a surfactant such as Polysorbate 80 at about 0.02%-2% by weight; carbomer copolymer (type A or type B) about 0.05% by weight; tonicity agent (glycerin or includes glycerin about 2.2% by weight; sodium citrate and tris buffer of pH 6.0-8.0; an oil (e.g., castor oil) in the amount of about 1.25% by weight, and acid/base solution to adjust the pH.

Yet another example of a formulation with its various components (w/w) is as follows: Carbopol 971 at about 0.1% to 0.4% by weight, poloxamer/tyloxapol at about 0.1% and 0.3% by weight; tonicity agent (glycerin or includes glycerin about 1% to 3% by weight; sodium citrate and tris buffer of pH 6.0-8.0; sodium EDTA in the amount of about 0.02% or less by weight.

Still another example of a formulation with its various components (w/w) is as follows: povidone at about 0.6% by weight, poloxamer/tyloxapol at about 0.1% and 0.25% by weight; tonicity agent (glycerine or includes glycerine about 1 to 3% by weight; sodium citrate and tris buffer of pH 6.0-8.0; sodium EDTA in the amount of about 0.02% or less by weight.

Still yet in another example, the oil-in-water nanoemulsion contains glycerin, a polymeric emulsifier (e.g., Acrylates/C₁₀₋₃₀ Alkyl Acrylate Cross-Polymer or Pemulen®), castor oil, a surfactant (e.g., polysorbate 80) and a buffering salt (e.g., sodium citrate dehydrate), and has a pH of 5 to 8. Preferably glycerin is present at a concentration of about 2.2% w/w, the polymeric emulsifier is present at a concentration of about 0.05% w/w, castor oil is present at a concentration of about 1.25% w/w, the surfactant is present at a concentration of about 1% w/w, and the buffering salt is present at a concentration of about 0.15% w/w. In addition, a small amount of Tris base (at about 0.03% w/w) can be present. The pH of the nanoemulsion can be adjusted using preferably HCl 0.1N and/or NaOH 0.1N as required. Water for injection q.s. is added.

One particular nanoemulsion formulation composition is shown in Table 1 below.

TABLE 1
An example of the composition of the invention.

| Ingredient | Range per g | | Per g | Other substitutes | Function |
|---|---|---|---|---|---|
| Castor Oil | 5-100 | mg | 12.5 mg | Olive oil, Oleic acid, etc. | Oil Phase |
| Polysorbate | 0.1 to 40 | mg | 10 mg | Polysorbate-20, | A component to both help |
| 80 | | | | Poloxamer 188 | facilitate the formation of the |
| | | | | | heterogeneous mixture and |
| | | | | | improve its stability. |
| Pemulen TR-2 | 0.1 to 2 | mg | 0.5 mg | N/A | Emulsion Stabilizer |
| Glycerin | 0-100 | mg | 22 mg | Trehalose, sorbitol, | Tonicity-Adjusting Agent |
| | | | | mannitol, | |
| | | | | xylitol | |
| Sodium | 0-20 | mg | 1.47 mg | Phosphate, tris, histidine, | Maintain pH |
| Citrate | | | | acetate, succinate | |
| Dihydrate | | | | | |
| Tris Base | 0-15 | mg | 1 mg | | Maintain pH |

| | | | | |
|---|---|---|---|---|
| Preservative | Optional | Optional | benzalkonium chloride | For multidose and nonsterile |
| | | | (BAK), stabilized | products |
| | | | oxychloro complex | |
| | | | (Purite) | |
| Base for pH | | | pH 5 to 8 | Maintain pH |
| Acid for pH | | | pH 5 to 8 | Maintain pH |
| Water for | | q.s. | | Vehicle |
| Injection | | | | |

The physical stability of these exemplary nanoemulsion formulations can be monitored. For example, the formulations are allowed to stand for a period of time (e.g., 6 months) at 20 to 25° C., and the heterogeneity sizes are measured. The heterogeneity sizes, within experimental error, should be identical at end of the test period to those measured right after the nanoemulsion is prepared thereby suggesting that there is no significant coalescence of the heterogeneity. Such results demonstrate that the nanoemulsion formulations so prepared have superior physical stability.

A nanoemulsion formulation of the present invention can be a finished dosage form that does not contain an active ingredient. Preferably, the dosage form of the invention is eye drops of the nanoemulsion formulations. The formulations of the present invention can be packaged in various package forms known in the field of topical ophthalmic. The formulations can be packaged in sterile single-use (unit dose) or multi-dose vials, preservative-free. In one embodiment, the formulation is packaged in sterile, preservative-free single-use packs or vials or containers (i.e., the unit dose vials). Each vial, for example as small as a 0.9 mL, may be made of low-density polyethylene so as to contain a small quantity of the formulation, e.g., 0.1-0.4 mL for a single use. This way, where the pharmaceutical composition is sterilized and contained in disposable single-dose containers for topical use in drop form, multiple vials in the form of a set of 30 vials, 60 vials and so on can be packaged in a tray with a lid, for example, a polypropylene tray with an aluminum peelable lid. The entire contents of each tray can be dispensed intact, and one vial or pack is used each time and immediately discarded after each use. For example, plastic ampules or vials or containers can be manufactured using blow-fill-seal (BFS) technology. The BFS processes may involve plastic extrusion, molding, aseptic filling, and hermetic sealing in one sequential operation and those processes are known in the art. In another embodiment, the formulation is packaged in multi-dose vials such that the materials can be dispensed as sterile at each time using specialized container/closure maintaining the sterility integrity. A number of new products are now available that utilize dispensers that incorporate unidirectional valves that allow multi-dose bottles to be unpreserved. In yet another embodiment, the formulation is packed in conventional vials/containers as sterile product for use multiple times before discarding it.

In another aspect, the invention provides methods of using the nanoemulsion for caring for cosmetic, dermatological or ophthalmological conditions. Preferably, the formulations of the present invention are used for the treatment of or relief from various ophthalmic conditions or eye disorders. In some embodiments, a method of improving or ameliorating the symptoms of ocular surface disorders is provided. An effective amount of the formulation of the present invention is administered topically to an eye of a subject or patient (e.g., a non-human mammal or a human) in need of the improvement or amelioration. An effective amount of the oil-in-water nanoemulsion is the amount sufficient to realize relief from the ocular surface disorder after the topical administration.

In particular, the invention provides a method for improving or ameliorating the symptoms of dry eye syndrome or from contact lens use by administering the nanoemulsions disclosed herein. There are two major classes of dry eye syndrome: (i) aqueous tear-deficient dry eye (ADDE) and (ii) evaporative dry eye (EDE). There are also cases of mixed mechanism dry eye (i.e., both ADDE and EDE). ADDE is primarily due to failure of lacrimal tear secretion. ADDE can be further subdivided into Sjogren syndrome dry eye (where the lacrimal and salivary glands are targeted by an autoimmune process, e.g., rheumatoid arthritis) and non-Sjogren's syndrome dry eye (lacrimal dysfunction, but the systemic autoimmune features of Sjogren's syndrome are excluded, e.g., age-related dry eye). In contrast, EDE is primarily due to excessive water loss from the exposed ocular surface in the presence of normal lacrimal secretory function. Its causes can be extrinsic (e.g., ocular surface disorder due to some extrinsic exposure, contact lens wear or vitamin A deficiency) or intrinsic (e.g., Meibomian gland dysfunction and disorders of eyelid aperture). Meibomian glands secrete a mixture of lipids and other components that form the outer layer of the preocular tear film. This lipid layer functions to decrease tear film evaporation. Meibomian gland dysfunction (MGD) leads to evaporative dry eye disease. One of the most well-recognized clinic finding in MGD is the presence of numerous telangiectatic blood vessels coursing across the eyelid margin. MGD can also accompany tear-deficient dry eye disease, as seen in ocular graft-versus-host-disease (oGVHD). Other specific dry eye syndromes that can be treated using compositions of the invention include keratoconjunctivitis, dry eye caused by conjunctivitis, dry eye caused by allergic conjunctivitis, dry eye caused by blepharitis, dry eye caused by keratitis, dry eye caused by dacryoadenitis, dry eye caused by ocular rosacea, dry eye caused by boehm syndrome, dry eye caused by conjunctivochalasis, dry eye caused by blepharoconjunctivitis, dry eye caused by blepharokeratoconjunctivitis, dry eye caused by superficial punctuate keratitis, dry eye caused by thygeson's superficial punctuate keratopathy, dry eye caused by oGVHD, Sjogren's dry eye syndrome, dry eye caused by Stevens-Johnson syndrome, MGD, dry eye caused by meibomian gland disease, vitamin A deficiency induced dry eye, pharmacological induced dry eye (i.e. hormone replacement therapy, blood pressure medication, antihistamine, antidepressants, anticholinergic medications, glaucoma medication, antihypertensives, diuretics, sedatives, isotretinoin, nasal decongestants, oral contraceptives, beta-blockers, phenothiazines, atropine, pain relieving opiates), pregnancy induced dry eye, LASIK surgery or refractive surgery induced dry eye, dry eye induced by collagen vascular diseases (i.e. systemic lupus erythematosus, Wegener's granulomatosis, rheumatoid arthritis, relapsing polychondritis), dry eye caused by the infiltration of the lacrimal glands by tumors or sarcoidosis, dry eye caused by postradiation fibrosis of tear producing glands, dry eye caused by lacrimal gland, meibomian gland, or goblet cell ablation, dry eye caused by sensory denervation, dry eye caused by thermal or chemical burns, dry eye caused by underlying diabetic conditions, dry eye caused by viral, fungal, or bacterial infection, dry eye caused by prolonged contact lens use, dry eye caused by eyelid disorders or injury to the eyelid (i.e. bulging eyes, drooping eyelid), dry eye caused by corneal dystrophy, dry eye caused by autoimmune disorders, age-induced dry eye, and a combination thereof. Preferably, the present invention provides a method of treating ocular surface abnormalities, defects, deficiencies, disorders, symptoms or diseases caused by contact lens solution or contact lens use.

The nanoemulsions can be used for treating, improving or ameliorating the symptoms of ocular disorders including dry eye syndrome by administering to a subject (e.g., a human patient) in need of such a treatment an effective amount of a given formulation, preferably a preservative-free, sterile filtered nanoemulsion having oil globules with a median size as disclosed herein. Preferred eye disorders for treatment include a dry eye syndrome (e.g., sjogren's syndrome, meibomian gland dysfunction and keratoconjunctivitis). Preferably, the nanoemulsion is administered topically to an eye of the subject.

The terms nanoemulsion, formulation, nanoemulsion formulation, placebo nanoemulsion or placebo have all been used interchangeably herein, and refer to nanoemulsions of the present invention not containing any active pharmaceutical ingredient(s). Unless otherwise specified, the ingredient amounts in the formulations of the present invention are presented in units of either % weight/volume (% w/v) and/or weight/weight (% w/w). As used herein, the term “about” is not intended to limit the scope of the invention but instead encompass the specified material, parameter or step as well as those that do not materially affect the basic and novel characteristics of the invention. When referring to a numeric value, the term “about” or “approximately” as used herein refers to being within an acceptable error range for the particular value as determined by one of ordinary skill in the art, which will depend in part on how the value is measured or determined (e.g., the limitations of the measurement system) or the degree of precision required for a particular purpose. For example, the term “about” can mean within 1 or more than 1 standard deviation, per the practice in the art. Alternatively, the term “about” when referring to a numerical value can mean±20%, typically ±10%, often ±5% and more often ±1% of the numerical value. In general, however, where particular values are described in the application and claims, unless otherwise stated, the term “about” means within an acceptable error range for the particular value. Moreover, any numerical value is to be understood to be within the one standard deviation unit per the practice in the art.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1. Nanoemulsion

A batch of a nanoemulsion was prepared, the final composition of which is presented in Table 2 below, according to the process flow below:
1. Oil phase: Mixed appropriate amounts of castor oil and polysorbate 80 until uniformity is obtained.
2. Aqueous phase: Mixed required amounts of Pemulen, water and glycerin until uniformity is obtained
3. Performed primary mixing of oil and aqueous phase mixtures from steps 1 and 2 understirring at 100-200 rpm for several minutes.
4. Performed high shear mixing at 3000-15000 rpm for 5-15 min depending on the manufacturing scale and at temperature of between 55-65° C.
5. Perform high-pressure microfludization of mixture from step 4 at 20000-30000 psi at a temperature of about 55-65° C.
6. Formulate nanoemulsion with other excipients like stabilizer, tonicity modifiers, buffer salts and pH adjusting solutions.
7. Perform sterile filtration and Confirm the ophthalmic solution properties via in-process testing

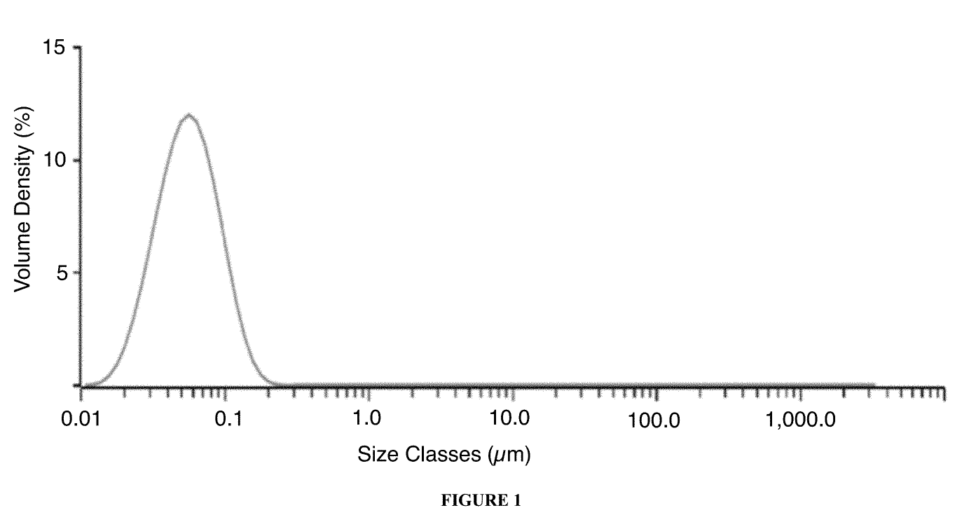

The nanoemulsion so prepared (without any active pharmaceutical ingredients added to it) was sterile filtered. The Viscosity of the placebo nanoemulsion prepared was 1.4 cP using Brookfield viscometer (Model-DV2TLVCJ) and Spindle (CP-52) at 20 rpm. The oil globules of the final nanoemulsion had a median size (Dv50) less than 100 nm. In particular, the median size was in the range of 40-80 nm. See FIG. 1.

TABLE 2
Final Composition of an exemplary nanoemulsion formulation (NanoE)

| Raw Material | Manufacturer | For 1 Kg |
|---|---|---|
| Glycerin Vegetable, | P&G (Superol KPO) | 22 g |
| USP/EP | | |
| Pemulen TR-2, NF | Lubrizol | 0.5 g |
| Castor Oil, USP/EP | Croda JSJ0470 | 12.5 g |
| Polysorbate 80, NF/EP | Croda SD43361 | 10 g |
| Sodium Citrate | Millipore - Sigma | 1.47 g |
| Dihydrate, USP/EP | Aldrich | |
| Tris Base, USP/EP | Millipore - Sigma | 0.3 g |
| | Aldrich | |
| HCl, 0.1N | Avantor | As Required to pH adjust |
| NaOH, 0.1N | Avantor | As Required to pH adjust |
| Water for Injection, | TRC | As Required to q.s. |
| USP/EP | | |

### Example 2. Efficacy of Nanoemulsion Formulation

Efficacy of NanoE (or ONE™) relative to a compositions containing lifitegrast (an active pharmaceutical ingredient) for the treatment of dry eye, an ocular disorder, is shown herein using an art recognized mouse model of dry eye disease (DED). The nanoemulsion administered is a preservative-free nanoemulsion. Commercial dry eye disease product containing lifitegrast was used for comparison.

For the treatment, C57BL/6 mice were exposed to a desiccating environment combined with transdermal administration of scopolamine for a period of two weeks. Treatments were started 1 day prior to exposure to the desiccating environment and throughout dry-eye disease induction.

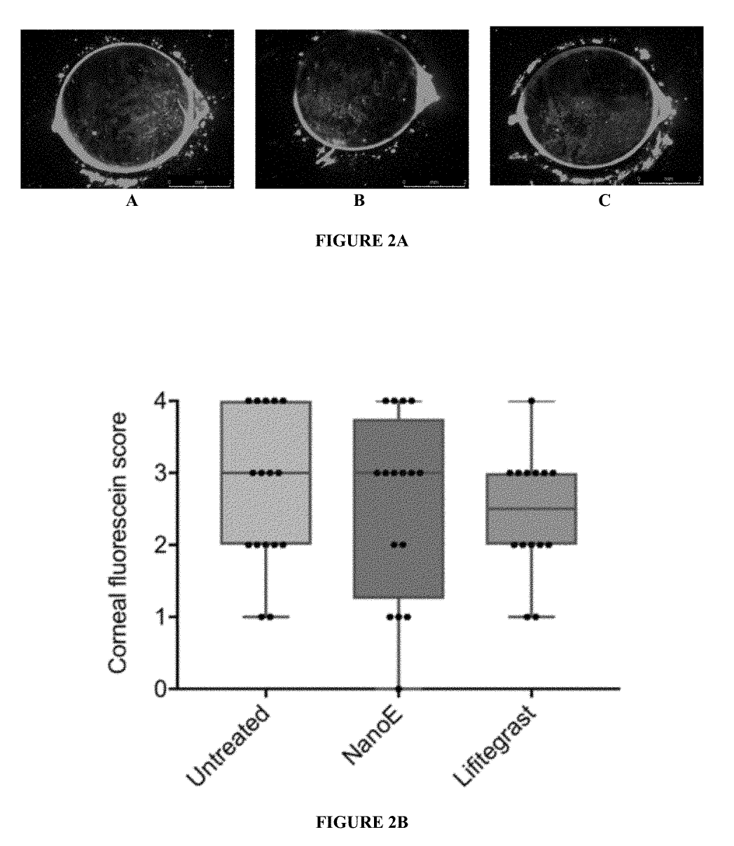

Treatment groups were as follows: (a) untreated; (b) NanoE (a nanoemulsion formulation, ONE′) only without any active pharmaceutical ingredient(s); (c) Xiidra® (lifitegrast ophthalmic solution) 5% lifitegrast. Each of these formulations ((b)-(c)) were administered topically at 10 μL per administration per eye twice daily. Corneal surface inflammation and damage was assessed by fluorescein staining. Lacrimal gland pathology was scored by qualitative assessment of the extent of immune cell infiltration and parenchymal damage based on H&E staining. The number of goblet cells in the conjunctiva was quantified using stereological counting of PAS-stained sections. FIGS. 2A and 2B show representative fluorescein staining scores. The NanoE group was comparable to the commercial lifitegrast and had improved response compared to untreated control. Fluorescein score is proportional to the level of ocular surface inflammation. These results were unexpected. Nanoemulsion formulations of the present invention can be advantageously used as over-the-counter artificial tears. The usefulness of the formulation as over-the-counter artificial tears is an advantage of the nanoemulsion of the present invention.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. An oil-in-water nanoemulsion comprising oil globules with a median size (Dv50) of about 100 nm or less, wherein said nanoemulsion is a sterile-filtered nanoemulsion and comprises an oil, an emulsion stabilizing polymer, a water soluble polymer, a surfactant, a tonicity modifier or stabilizer, or a combination thereof.

2. The oil-in-water nanoemulsion of claim 1, wherein said nanoemulsion has anti-inflammatory activity on the ocular surface as determined by an improved corneal fluorescein staining (CFS).

3. The oil-in-water nanoemulsion of claim 1, wherein the oil is castor oil, corn oil, olive oil or oleic acid, or a combination thereof.

4. The oil-in-water nanoemulsion of claim 1, wherein the surfactant comprises polysorbate 80, the emulsion stabilizing polymer, a polyol or a combination thereof.

5. The oil-in-water nanoemulsion of claim 1, wherein the tonicity modifier or stabilizer is selected from the group consisting of a polyol, a non-reducing disaccharide, and a combination thereof.

6. The oil-in-water nanoemulsion of claim 1, wherein the emulsion stabilizing polymer comprises carbomer copolymer type A or type B.

7. The oil-in-water nanoemulsion of claim 1, wherein the oil globules have a median size (Dv50) ranging from about 20 to about 80 nm.

8. The oil-in-water nanoemulsion of claim 7, wherein the oil globules have a median size (Dv50) ranging from about 40 to about 60 nm.

9. The oil-in-water nanoemulsion of claim 7, wherein said nanoemulsion has a viscosity ranging from 1 to 10 cP when measured at 32° C. or lower.

10. The oil-in-water nanoemulsion of claim 7, wherein said nanoemulsion comprises glycerin, a polymeric emulsifier, castor oil, a surfactant and a buffering salt, and has a pH ranging from pH of about 5 to pH of about 8.

11. The oil-in-water nanoemulsion of claim 10, wherein said glycerin is present at a concentration of about 2.2% w/w, said polymeric emulsifier is present at a concentration of about 0.05% w/w, said castor oil is present at a concentration of about 1.25% w/w, said surfactant is present at a concentration of about 1% w/w, and said buffering salt is present at a concentration of about 0.15% w/w.

12. The oil-in-water nanoemulsion of claim 7, further comprising a cosmetically, dermatologically, or ophthalmologically acceptable carriers.

13. A method of preparing an oil-in-water nanoemulsion of claim 1, comprising:
mixing an aqueous phase and an oil phase with vigorous stirring at a temperature ranging from 55 to 65° C.;
homogenizing the mixture at a temperature ranging from 55 to 65° C.; and
subjecting said homogenized mixture to high pressure shearing at a temperature ranging from 55 to 65° C. and at a pressure ranging from 20000 to 30000 psi to produce the oil-in-water nanoemulsion of claim 1.

14. The method of claim 13, wherein said step of homogenizing the mixture is conducted at 3000 to 15000 rpm.

15. The method of claim 13 further comprising the step of filtering said oil-in-water nanoemulsion of claim 1 through a 0.22 μm filter to obtain said sterile oil-in-water nanoemulsion

16. A method of treating an ocular surface disorder, the method comprising administering topically to an eye of a subject in need of such a treatment an effective amount of an oil-in-water nanoemulsion of claim 1, wherein the subject is a non-human mammal or a human.

17. The method of claim 16, wherein said ocular surface disorders is a dry eye syndrome.

18. The method of claim 16, wherein said oil-in-water nanoemulsion is preservative-free.

19. The method of claim 16, wherein said oil-in-water nanoemulsion has anti-inflammatory activity.

20. The method of claim 16, wherein a median size (Dv50) of oil globules in said oil-in-water nanoemulsion ranges from 20 to 80 nm.
